(19) United States
(12) Patent Application Publication
(10) Pub. No.: US 2006/0079933 A1
(43) Pub. Date: Apr. 13, 2006

(54) Latching mechanism for forceps

(76) Inventors: Dylan Hushka, Boulder, CO (US); David M. Garrison, Longmont, CO (US)

Correspondence Address: UNITED STATES SURGICAL,;A DIVISION OF TYCO HEALTHCARE GROUP LP, 150 GLOVER AVENUE, NORWALK, CT, 06856, US

(21) Appl. No.: 11/232,256

(22) Filed: Sep. 21, 2005

Related U.S. Application Data
(60) Provisional application No. 60/616,973, filed on Oct. 8, 2004.

(51) Int. Cl. A61B 17/28

(52) U.S. Cl. 606/205

(57) ABSTRACT

The present disclosure relates to a surgical instrument which includes a shaft having a proximal end and a distal end and a housing connected to the proximal end of the shaft. The instrument may further include an actuation assembly having a first lever and a second lever, each pivotally connected to the housing. An end effector assembly is operatively connected to the distal end of the shaft and includes a pair of opposing jaw members. The instrument may further include a drive rod slidably disposed within the shaft and connected to the actuation assembly, the drive rod being operable by the actuation assembly to actuate the opposing jaw members between open and closed positions. A latching mechanism may be provided which is operatively associated with the actuation assembly and drive rod for maintaining the jaw members in the closed position.

### CROSS REFERENCE TO RELATED APPLICATIONS

This application claims priority from U.S. Provisional Application Ser. No. 60/616,973 filed on Oct. 8, 2004, the contents of which are hereby incorporated by reference in their entirety.

### BACKGROUND

1. Technical Field

The present disclosure relates to surgical instruments including hemostat-type and forceps-type surgical instruments for performing surgical functions and, more particularly, hemostat-type or forceps-type surgical instruments including improved latching mechanisms for opening and closing or otherwise operating an end effector of the surgical instrument.

2. Background of Related Art

A hemostat or forceps is a simple plier-like tool which uses mechanical action between its jaws to constrict vessels and is commonly used in open surgical procedures to grasp, dissect and/or clamp tissue. Electrosurgical forceps utilize both mechanical clamping action and electrical energy to effect hemostasis by heating the tissue and blood vessels to coagulate, cauterize and/or seal tissue.

Typically, the hemostat or forceps includes an interlocking ratchet between the handles so that the device may be clamped and/or locked into place. It is not uncommon for many hemostats or forceps to be used in a typical open-surgical procedure. Once vascular tissue has been clamped with a hemostat or forceps, it is common for a surgeon to tie a suture around the tissue to close it off permanently prior to removing the hemostat. Several hemostats may be left in the surgical field until the surgeon has the opportunity to tie a suture around each section of clamped tissue.

The interlocking ratchet typically requires one lever or arm of the hemostat or forceps to travel over the other, thereby requiring an exerted torque by the user. Additionally, typical interlocking ratchets require an unlatching torque to open the device.

A continuing need exists for an improved hemostats or forceps which is easier to operate by the user, which requires less torque by the user in order to close the device and which is relatively simple to manufacture.

A need also exists for improved hemostats or forceps which eliminates the unlatching torque required to open the device, thereby making the device more controllable in delicate operations and thereby reducing fatigue during periods of continued use.

### SUMMARY

The present disclosure relates to a surgical instrument comprising a shaft having a proximal end and a distal end and a housing connected to the proximal end of the shaft. The instrument may further include an actuation assembly having a first and second lever, each of the first and second levers being pivotally connected to the housing. An end effector assembly which is operatively connected to the distal end of the shaft is provided and includes a pair of opposing jaw members. The instrument may further include a drive rod slidably disposed within the shaft and connected to the actuation assembly, the drive rod being operable by with the actuation assembly to actuate the opposing jaw members between open and closed positions. A latching mechanism may be provided which is operatively associated with the actuation assembly and drive rod for maintaining the jaw members in the closed position.

In one embodiment of the present disclosure the surgical instrument may include a rotating assembly operatively associated with the drive rod for rotating the end effector assembly. Moreover, it is envisioned for the instrument to be either fully or partially disposable.

In another embodiment of the present disclosure the instrument may include a first link and a second link, the first link having a proximal end pivotally connected to the first lever and a distal end pivotally connected to the proximal end of the drive rod. Similarly the second link would include a proximal end pivotally connected to the second lever and a distal end pivotally connected to the proximal end of the drive rod. In some embodiments either the first or second link is of a greater length than the other link.

Moreover, the instrument may include a first stem and a second stem connecting the proximal ends of the first and second links to the proximal ends of the first and second levers respectively.

It is envisioned for the instrument to further include a biasing member disposed between a distal surface of a flange formed on the drive rod and a fixed proximal surface provided on one of the housing and the shaft. In one embodiment the biasing member may be a compression spring.

In yet another embodiment of the present disclosure the instrument may further include a first linkage and a second linkage, the first linkage connecting the first lever with the drive rod and the second linkage connecting the second lever with the drive rod.

In certain embodiments of the present disclosure a release lever may be included which is operatively associated with at least one of the first and second links. The release lever may include a head portion configured to be selectively engaged by a catch located adjacent to the actuation assembly when the actuation assembly is in a closed position. Furthermore, the release lever may include a cantilevered arm, the arm defining a catch and being configured to facilitate locking and unlocking of the apparatus.

In another embodiment the apparatus further comprises a biasing member operatively connected between a stem extending from one of the first and second levers and a second link pivotally connected to the stem and a first link pivotally extending from the other of the first and second levers, the biasing member being configured to at least one of maintain and return the actuation assembly to the closed position. Moreover, it is envisioned for the biasing member to be a tension spring.

In yet another embodiment of the present disclosure a shaft having a proximal end and a distal end and a housing connected to the proximal end of the shaft is provided. The instrument may further include an actuation assembly having a first lever and a second lever, each lever being pivotably connected to the housing. An end effector assembly which is operatively connected to the distal end of the shaft is provided which includes a pair of opposing jaw members. The instrument may further include a drive rod slidably disposed within the shaft and connected to the actuation assembly, the drive rod being operable by the actuation assembly to actuate the opposing jaw members between open and closed positions. The instrument may also include a first link and a second link each having a distal end pivotally connected to the first and second levers respectively. A mandrel may be fixedly mounted to the drive rod, proximal ends of the first and second links being pivotally attached to the mandrel. A biasing member may be provided which is configured to bias the actuation assembly to one of an open and closed condition.

In some embodiments of the present disclosure a release trigger may be included which is pivotally connected to at least one of the first and second levers, the release trigger being dimensioned to allow for the latching mechanism or instrument to transition between an open position and a closed position.

### BRIEF DESCRIPTION OF THE DRAWINGS

Various embodiments of the subject devices and latching mechanisms are described herein with reference to the drawings wherein:

FIG. 1 is a perspective view of a hemostat-type or forceps-type surgical instrument in accordance with an embodiment of the present disclosure;

FIG. 2 is an enlarged, perspective view of the indicated area of detail of FIG. 1, showing an enhanced view of an end effector assembly of the surgical instrument of FIG. 1;

FIG. 3 is a schematic, side cross-sectional view of the housing of the surgical instrument of FIG. 1, illustrating a latching mechanism according to an embodiment of the present disclosure while in an open or unlatched condition;

FIG. 4 is a schematic, side cross-sectional view of the housing of the surgical instrument of FIG. 1, illustrating the latching mechanism of FIG. 3 while in a closed or latched condition;

FIG. 5 is a schematic, side cross-sectional view of the housing of the surgical instrument of FIG. 1, illustrating a latching mechanism according to another embodiment of the present disclosure while in an open or unlatched condition;

FIG. 6 is a schematic, side cross-sectional view of the housing of the surgical instrument of FIG. 1, illustrating the latching mechanism of FIG. 5 while in a closed or latched condition;

FIG. 7 is a schematic, side cross-sectional view of the housing of the surgical instrument of FIG. 1, illustrating a latching mechanism according to yet another embodiment of the present disclosure while in a closed or latched condition;

FIG. 8 is a schematic, side cross-sectional view of the housing of the surgical instrument of FIG. 1, illustrating a latching mechanism according to a further embodiment of the present disclosure while in a closed or latched condition;

FIG. 9 is a schematic, side cross-sectional view of the housing of the surgical instrument of FIG. 1, illustrating a latching mechanism according to another embodiment of the present disclosure while in an open or unlatched condition;

FIG. 10 is a schematic, side cross-sectional view of the housing of the surgical instrument of FIG. 1, illustrating the latching mechanism of FIG. 9 while in a closed or latched condition;

FIG. 11 is a schematic illustration of a release trigger for any of the latching mechanisms of FIGS. 3-10, shown in a clamped position; and

FIG. 12 is a schematic illustration of the release trigger of FIG. 11, shown in an unclamped position.

### DETAILED DESCRIPTION

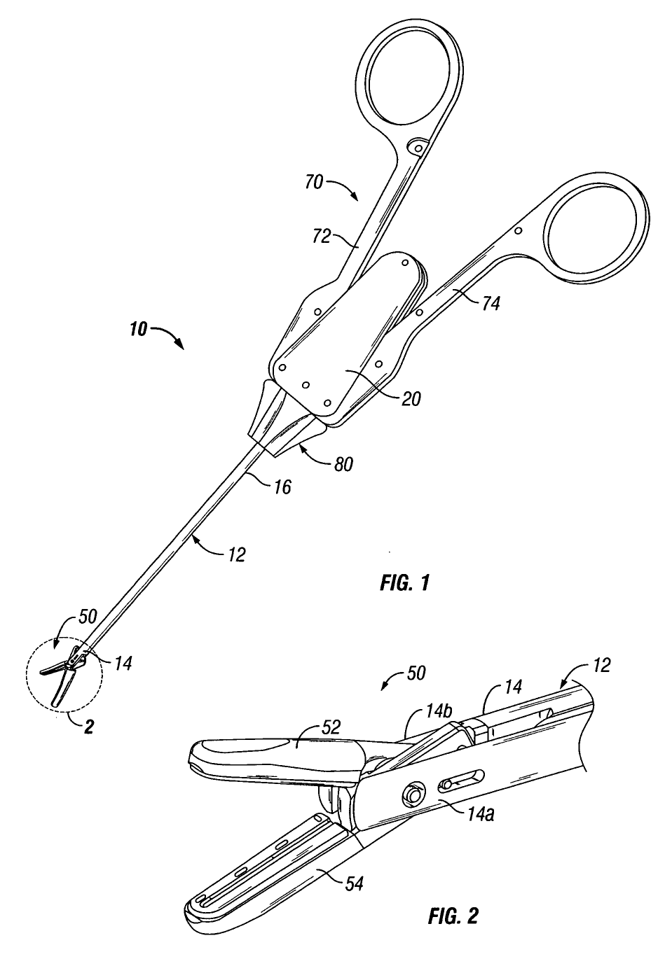

Referring now to FIGS. 1 and 2, one embodiment of a hemostat-type or forceps-type surgical instrument, in accordance with the present disclosure is generally designated as 10. In the drawings and in the description which follows, the term “proximal”, as is traditional, will refer to the end of surgical instrument 10 which is closer to the user, while the term “distal” will refer to the end of surgical instrument 10 which is further from the user.

As seen in FIG. 1, surgical instrument 10 generally includes a housing 20, an actuating assembly 70 operatively associated with housing 20, and an end effector assembly 50 operatively associated with housing 20 and actuation assembly 70. End effector assembly 50 may function to grasp, seal and divide tubular vessels and vascular tissue. Although surgical instrument 10 is shown in FIGS. 1 and 2 as being configured for use in connection with endoscopic surgical procedures, an open-type surgical instrument (not shown) is also contemplated for use in connection with traditional open surgical procedures. For the purposes herein, the endoscopic version is discussed in detail, however, it is contemplated that open-type surgical instruments may also include the same or similar operating components and features as those described below.

In particular, as seen in FIGS. 1 and 2, surgical instrument 10 includes a shaft 12 having a distal end 14 configured to mechanically engage end effector assembly 50 and a proximal end 16 which mechanically engages housing 20. Preferably, as seen in FIG. 2, distal end 14 of shaft 12 is bifurcated to form ends 14a and 14b which are dimensioned to matingly receive end effector assembly 50.

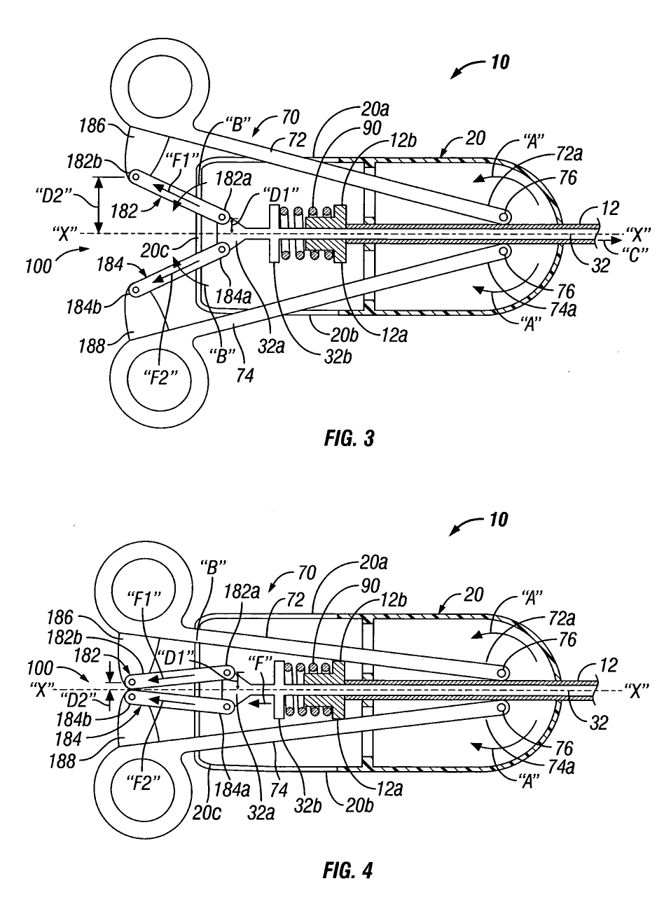

As seen in FIGS. 1, 3 and 4, actuation assembly 70 includes a first lever 72 and a second lever 74, each pivotably connected to housing 20. First and second levers 72, 74 of actuation assembly 70 are movable relative to housing 20 as explained in more detail below with respect to the operation of surgical instrument 10. Rotating assembly 80 is preferably attached to a distal end of housing 20 and is rotatable approximately 180° in either direction about a longitudinal axis “X” (see FIG. 3).

As mentioned above and as seen in FIG. 2, end effector assembly 50 is attached to distal end 14 of shaft 12 and includes a pair of opposing jaw members 52 and 54. Actuation assembly 70 is ultimately connected to a drive rod 32 (see FIGS. 3 and 4) which, together, mechanically cooperate to impart movement of jaw members 52 and 54 from an open position wherein jaw members 52, 54 are disposed in spaced relation relative to one another, to a clamping or closed position wherein jaw members 52, 54 cooperate to grasp tissue therebetween. As seen in FIGS. 3 and 4, drive rod 32 is slidably disposed within shaft 12.

It is envisioned that surgical instrument 10 may be designed such that it is fully or partially disposable depending upon a particular purpose or to achieve a particular result. For example, end effector assembly 50 may be selectively and releasably engageable with distal end 14 of shaft 12 and/or proximal end 16 of shaft 12 may be selectively and releasably engageable with housing 20. In either of these two instances, surgical instrument 10 wound be considered “partially disposable” or “reposable”, i.e., a new or different end effector assembly 50 selectively replaces the old end effector assembly 50 as needed.

Turning now to the more detailed features of the present disclosure as described with respect to FIGS. 3 and 4, distal ends 72a, 74a of respective first and second levers 72, 74 of actuation assembly 70, are pivotally connected within housing 20 by pivot pins 76. Desirably, pivot pins 76 are located in close proximity to the longitudinal “X” axis. Proximal ends 72b and 74b of respective first and second levers 72, 74 of actuation assembly 70 extend proximally out of housing 20 through respective side slots 20a and 20b when actuation assembly 70 is in an open condition and desirably proximally out of an open rear slot 20c of housing 20 when actuation assembly 70 is in a closed condition.

As seen in FIGS. 3 and 4, according to one embodiment of the present disclosure, surgical instrument 10 includes a latching mechanism 100 operatively associated with actuation assembly 70 and drive rod 32. Latching mechanism 100 includes a first link 182 having a proximal end 182b pivotally connected to first lever 72 and a distal end 182a pivotally connected to a proximal end 32a of drive rod 32. Additionally, latch mechanism 100 includes a second link 184 having a proximal end 184b pivotally connected to second lever 74 and a distal end 184a also pivotally connected to proximal end 32a of drive rod 32.

Also as seen in FIGS. 3 and 4, proximal end 32a of drive rod 32 is enlarged. Likewise, distal ends 182a and 184a of respective first and second links 182 and 184 are pivotally connected to enlarged proximal end 32a such that distal ends 182a and 184a are spaced a radial distance “D1” away from the longitudinal “X” axis. As seen in FIG. 4, each lever 72 and 74 includes a respective stem 186 and 188 extending therefrom, preferably, in the direction of the longitudinal “X” axis. Desirably, proximal ends 182b and 184b of respective first and second links 182 and 184 are pivotally connected to respective stems 186 and 188 such that proximal ends 182b and 184b are spaced a distance “D2” from one another when actuation assembly 70 is in the closed condition.

Desirably, when actuation assembly 70 is in the closed condition, distance “D2” is less than distance “D1”.

Desirably, actuation assembly 70 is biased to the open condition by a biasing member 90 or the like. Preferably, biasing member 90 is in the form of a compression spring disposed between a distal surface of a flange 32b formed on drive rod 32 and a fixed proximal surface 12b formed in housing 20 or provided on shaft 12. For example, the fixed proximal surface 12b may be formed with a flange 12a which may be integrally formed at or may be secured to a proximal end 16 of shaft 12.

In use, as first and second levers 72 and 74 are approximated toward one another and/or toward the longitudinal “X” axis, first and second levers 72 and 74 pivot about pivot pins 76, in the direction of arrows “A”. As first and second levers 72 and 74 are approximated, proximal ends 182b and 184b of first and second links 182 and 184 are also approximated toward or pivoted toward one another and/or the longitudinal “X” axis, as indicated by arrows “B”. Since links 182 and 184 are pivotally connected to enlarged proximal end 32a of drive rod 32 and act on drive rod 32, approximation of links 182 and 184 results in drive rod 32 being driven in a distal direction, as indicated by arrow “C”.

As seen in FIG. 4, when actuation assembly 70 and/or latch mechanism 100 is in the fully closed condition, proximal ends 182b and 184b of first and second levers are spaced a distance “D2” relative to one another, while distal ends 182a and 184a are maintained at a distance “D1” relative to one another. Since distance “D2” is less than distance “D1” when actuation assembly 70 and/or latch mechanism 100 is in the fully closed position, and since biasing means 90 exerts a force “F” in a proximal direction on distal ends 182a and 184a of links 182 and 184, proximal ends 182b and 184b of links 182 and 184 will be maintained in the approximated position and actuation assembly 70 and/or latch mechanism 100 will be maintained in the closed position.

Desirably, distal movement of drive rod 32 relative to housing 20 and/or shaft 12 results in opening or closing of end effector assembly 50. As mentioned above, approximation of first and second levers 72 and 74 results in compression of biasing member 90, locking of latch mechanism 100 and maintenance of actuation assembly 70 in the closed condition. Accordingly, upon opening latch mechanism 100, first and second levers 72 and 74 are separated from one another such that the distance “D2” between proximal ends 182b and 184b of links 182 and 184 increases until distance “D2” is greater than distance “D1” between distal ends 182a, 184a of links 182 and 184. At such time, force “F” of biasing member 90 moves drive rod 32 in a proximal direction thereby opening or closing end effector assembly 50, further separating levers 72 and 74, and further opening latch mechanism 100.

Latching mechanism 100 including levers 72 and 74 function like an over-center latching or toggle mechanism. In other words, biasing member 90 transmits a respective force “F1 and F2” to each of first and second links 182 and 184. Accordingly, when proximal ends 182b and 184b are spaced a distance “D2” from one another which is greater than the distance “D1” between distal ends 182a and 184a of first and second links 182 and 184, first and second forces “F1 and F2” push first and second levers 72 and 74 away from one another. However, when proximal ends 182b and 184b are spaced a distance “D2” from one another which is less than the distance “D1” between distal ends 182a and 184a of first and second links 182 and 184, first and second forces “F1 and F2” push first and second levers 72 and 74 towards one another, thereby locking actuation assembly 70. In this manner, a relatively small force is required to begin opening of actuation assembly 70 until distance “D2” becomes greater than distance “D1” at which time forces “F1 and F2” will push first and second levers 72 and 74 apart.

Various forces and feels for actuation assembly 70 may be achieved by modifying the dimensions and proportions of the components of latching mechanism 100. For example, the feel and operation of actuation assembly 70 may be modified by increasing or decreasing the distance “D1”; by increasing or decreasing the length of first and second links 182 and 184; by increasing or decreasing the spring constant of biasing member 90; and/or by moving the location of where proximal ends 182b and 184b of first and second links 182 and 184 pivotally attach to respective first and second levers 72 and 74.

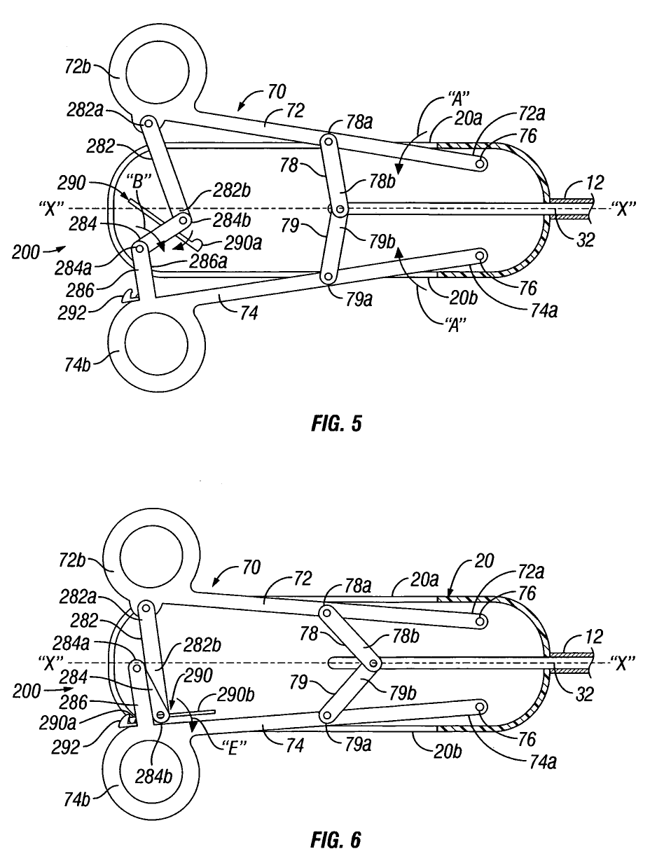

Turning now to FIGS. 5 and 6, a latching mechanism in accordance with another embodiment of the present disclosure is generally designated as 200. Latching mechanism 200 includes a first link 282 having a first end 282a pivotally connected to first lever 72 near a proximal end 72b thereof, and a second link 284 having a first end 284a pivotally connected to second lever 74 near a proximal end 74b thereof. Preferably, first link 282 includes a second end 282b which is pivotally connected to a second end 284b of second link 284.

Latching mechanism 200 includes a stem 286 extending from one of the first or second levers 72, 74, (e.g., from second lever 74 as shown in FIGS. 5 and 6). Stem 286 preferably extends in a direction towards first lever 72. Accordingly, second end 282b of second link 282 is pivotally connected to a distal end 286a of stem 286.

Desirably, latch mechanism 200 includes a lock/release lever 290 operatively associated with second link 284, and a catch or stop 292 formed on second lever 74. As will be described in greater detail below, catch 292 selectively engages an enlarged head portion 290a of lock/release lever 290 when actuation assembly 70 is in the closed condition (i.e., latch mechanism 200 is in the closed condition) to thereby maintain actuation assembly 70 and latch mechanism 200 in the closed condition and thereby lock actuation assembly 70.

As seen in FIGS. 5 and 6, actuation assembly 70 may include a first linkage 78 having a first end 78a pivotally connected to first lever 72 and a second linkage 79 having a first end 79a pivotally connected to second lever 74. Each linkage 78 and 79 includes a second end 78b and 79b, respectively, which are pivotally connected to proximal end 32a of drive rod 32. Desirably, second ends 78b and 79b of linkages 78 and 79 are located distally of first ends 78a and 79a. In this manner, as first ends 78a, 79a of linkages 78 and 79 are approximated toward one another upon approximation or squeezing of first and second levers 72 and 74, second ends 78b and 79b of linkages 78 and 79 move drive rod 32 in a distal direction relative to housing 20 and shaft 12. Additionally, as first ends 78a, 79a of linkages 78 and 79 are separated from one another upon opening of first and second levers 72 and 74, second ends 78b and 79b of linkages 78 and 79 move drive rod 32 in a proximal direction relative to housing 20 and shaft 12.

With continued reference to FIGS. 5 and 6, a method of operation of linkage 200 is shown and described. In use, as first and second levers 72, 74 are approximated toward one another and/or toward the longitudinal “X” axis, proximal ends 72b and 74b of first and second levers 72 and 74 pivot about pivot pins 76, in the direction of arrows “A”. As proximal ends 72b, 74b of first and second levers 72, 74 are approximated, second end 284b of second link 284 is pivoted about distal end 286a of stem 286, as indicated by arrow “B”.

As second link 284 is pivoted about distal end 286a of stem 286, detent 290 is also pivoted about distal end 286a of stem 286 until head portion 290a of lock/release lever 290 selectively engages catch 292. With head portion 290a of lock/release lever 290 engaged with catch 292, actuation assembly 70 and latch mechanism 200 are maintained in the closed condition and actuation assembly 70 is locked. It is envisioned that catch 292 may include a plurality of engagement locations (not shown) for engagement of head portion 290a of lock/release lever 290. In this manner, for example, engagement of head portion 290a of lock/release lever 290 with a distal-most engagement location results in a relatively smaller clamping force being transmitted to or created by end effector assembly 50. Additionally, engagement of head portion 290a of lock/release lever 290 with a proximal-most engagement location results in a relatively larger clamping force being transmitted to or created by end effector assembly 50.

Actuation assembly 70 and latch mechanism 200 may be un-locked or opened by actuating proximal end 290b of lock/release lever 290 (e.g., by pressing proximal end 290b in the direction of arrow “E”) to thereby disengage or release head portion 290a of lock/release lever 290 from catch 292. Upon release of head portion 290a of lock/release lever 290 from catch 292, first and second levers 72 and 74 may be separated, thereby separating first ends 78a and 79a of linkages 78 and 79 and moving drive rod 32 in a proximal direction relative to housing 20 and shaft 12. In so doing, end effector assembly 50 is either opened or closed.

In accordance with the present disclosure, and as described herein, latch mechanism 200 functions as an over-center latch or toggle mechanism, similar to latch mechanism 100 described above.

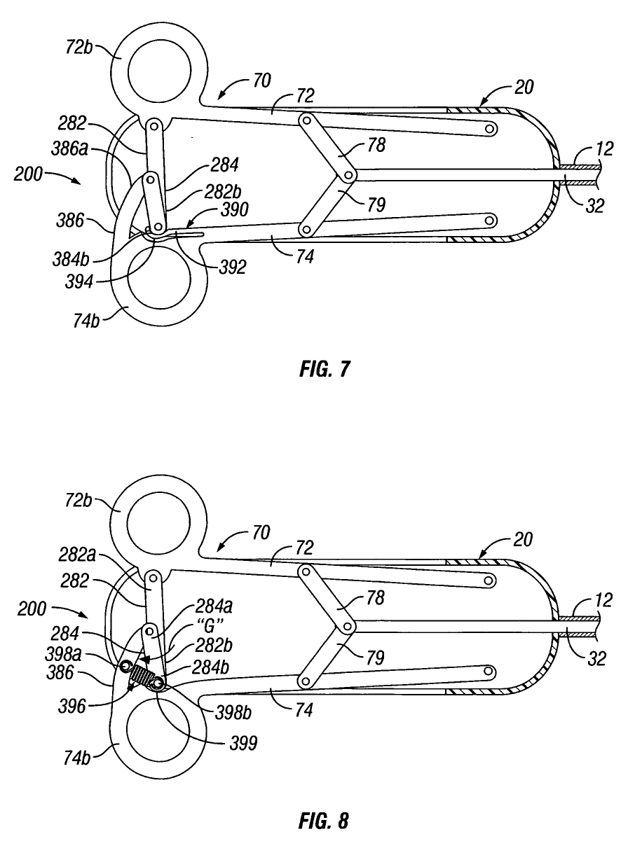

As seen in FIG. 7, latch mechanism 200 may include an alternate lock/release lever 390. Lock/release lever 390 includes a cantilevered arm 392 which extends from second lever 74 which is configured to deflect to facilitate locking and unlocking of the forceps. A distal end of arm 392 defines a catch 394 formed therein. Catch 394 is configured to selectively receive at least one second end 282b or 284b of first or second link 282, 284 therein, when actuation assembly 70 is in the closed position. Second lever 74 includes a stem 386 extending therefrom, preferably in a distal direction and in a direction toward first lever 72.

In use, as proximal ends 72b and 74b of first and second levers 72 and 74 are approximated towards one another, second end 284b of second link 284 is pivoted about distal end 386a of stem 386 until second end 284b of second link 284 selectively engages or is received in catch 394 of detent 390. With second end 284b of second link 284 engaged with catch 394 actuation assembly 70 is in a locked condition. In order to un-lock actuation assembly 70, arm 392 of detent 390 is deflected, in the direction of arrow “E” (see FIG. 6) to thereby release second end 284b of second link 284 from catch 394.

As seen in FIG. 8, in an alternate embodiment, latch mechanism 200 may include a biasing member 396 operatively connected between stem 386 and second link 284. Preferably, biasing member 396 is a tension spring or the like. One end of biasing member 396 is operatively connected to a first boss 398a provided on stem 386 and a second end of biasing member 396 is operatively connected to a second boss 398b provided on second link 284, preferably located at the pivot point between second ends 282b and 284b of first an second links 282, 284. Biasing member 396 maintains and/or returns actuation assembly 70 to the closed or locked position.

Additionally, a stop 399 may be formed in second lever 74 for preventing rotation, in the direction of arrow “G”, of second end 284b of second link 284 beyond stop 399. In this manner, the range of motion and rotation of second end 284b of second link 284 is defined.

In use, as proximal ends 72b and 74b of first and second levers 72 and 72 are separated from one another, second end 284b of second link 284 is pivoted about distal end 386a of stem 386 thereby biasing and/or elongating biasing member 396. As first and second levers 72 and 74 are separated from one another, first ends 78a and 79a (see FIGS. 5 and 6) of linkages 78 and 79 are separated from one another, thereby moving drive rod 32 in a proximal direction relative to housing 20 and shaft 12. In so doing, end effector assembly 50 is either opened or closed.

When proximal end 72b of first lever 72 and/or proximal end 74b of second lever 74 is released, biasing member 396 contracts and returns second end 284b of second link 284 against stop 399 and returns actuation assembly 70 to the closed condition.

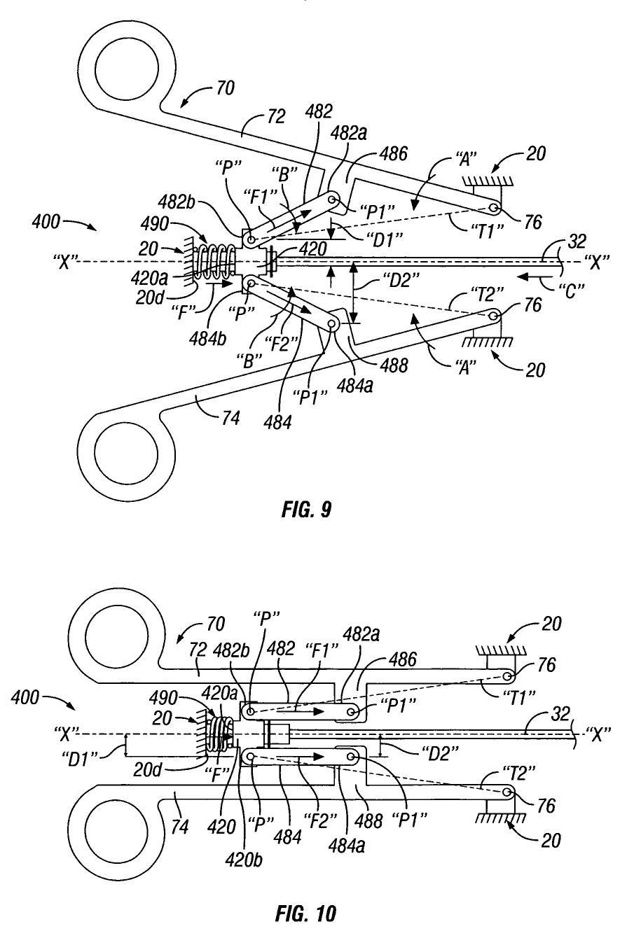

Turning now to FIGS. 9 and 10, a linkage mechanism according to another embodiment of the present disclosure is shown as 400. Linkage mechanism 400 is similar to linkage mechanism 100 and will only be discussed in detail to the extent necessary to identify differences in construction and operation.

As seen in FIGS. 9 and 10, latching mechanism 400 includes a first link 482 having a distal end 482a pivotally connected to first lever 72 and a proximal end 482b pivotally connected to a mandrel 420. Additionally, latch mechanism 400 includes a second link 484 having a distal end 484a pivotally connected to second lever 74 and a proximal end 484b pivotally connected to mandrel 420. Mandrel 420 is fixedly mounted to drive rod 32. Accordingly, distal or proximal movement of mandrel 420 results in corresponding distal or proximal movement of drive rod 32. Proximal ends 482b and 484b of first and second links 482 and 484 are spaced a radial distance “D1” away from the longitudinal “X” axis.

Each lever 72 and 74 includes a respective stem 486 and 488 extending therefrom, preferably, in the direction of the longitudinal “X” axis. Desirably, distal ends 482a and 484a are pivotally connected to respective stems 486 and 488 such that distal ends 482a and 484a are spaced a distance “D2” from one another when actuation assembly 70 is in the closed condition. When actuation assembly 70 is in the closed condition, distance “D2” is less than distance “D1”.

Desirably, actuation assembly 70 is biased to the open condition by a biasing member 490 or the like. Preferably, biasing member 490 is in the form of a compression spring disposed between a proximal surface 420a of mandrel 420 and a fixed surface 20d provided in housing 20.

In use, as first and second levers 72 and 74 are approximated toward one another and/or toward the longitudinal “X” axis, first and second levers 72 and 74 pivot about pins 76, in the direction of arrows “A”. As first and second levers 72 and 74 are approximated, distal ends 482a and 482b of first and second links are also approximated toward or pivoted toward one another and/or the longitudinal “X” axis, as indicated by arrow “B”. Since first and second links 482 and 484 are pivotally connected to mandrel 420, approximation of distal ends 482a and 484a of first and second links 482 and 484 results in drive rod 32 being driven in a proximal direction, as indicated by arrow “C”.

As seen in FIG. 10, when actuation assembly 70 and/or latch mechanism 400 is in the fully closed condition, distal ends 482a and 484a of first and second levers 72 and 74 are spaced a distance “D2” relative to one another, while proximal ends 482b and 484b of first and second links 482 and 484 are maintained at a distance “D1” relative to one another. Since distance “D2” is less than distance “D1” when actuation assembly 70 and/or latch mechanism 400 is in the fully closed position, and since biasing means 490 exerts a force “F” in a distal direction on proximal ends 482b and 484b of first and second links 482 and 484, distal ends 482a and 484a of first and second links 482 and 484 will be maintained in the approximated position and actuation assembly 70 and/or latch mechanism 400 will be maintained in the closed position.

Actuation assembly 70 and latch mechanism 400 is opened by separating first and second levers 72 and 74 from one another until the distance “D2” between distal ends 482a and 484a of first and second links 482 and 484 is greater than the distance “D1” between proximal ends 482b and 484b of first and second links 482 and 484. At such time, force “F” of biasing member 490 moves drive rod 32 in a distal direction thereby opening or closing end effector assembly 50, further separating first and second levers 72 and 74, and further opening latch mechanism 400.

Described differently, latching mechanism 400 includes a first transition line “T1” extending between pivot point 76 of first lever 72 and pivot point “P” of proximal end 482b of first link 482, and a second transition line “T2” extending between pivot point 76 of second lever 74 and pivot point “P” of proximal end 484b of second link 484. Transition lines “T1 and T2” define the line across which latching mechanism 400 transitions from self-opening to self-closing.

Accordingly, when the pivot point “P1” between distal end 482a of first link 482 and first lever 72 and/or the pivot point “P1” between distal end 484a of second link 484 and second lever 74 is positioned radially outward of transition lines “T1 and T2”, biasing member 490 acts to maintain latching mechanism 400 and actuation assembly 70 in the open condition. In particular, when pivot points “P1” are located radially outward of transition lines “T1 and T2”, biasing member 490 transmits forces “F1 and F2” along respective first and second links 482 and 484 which tend to force respective first and second levers 72 and 74 radially outward. Additionally, when the pivot point “P1” between distal end 482a of first link 482 and first lever 72 and/or the pivot point “P1” between distal end 484a of second link 484 and second lever 74 is positioned radially inward of transition lines “T1 and T2”, biasing member 490 acts to maintain latching mechanism 400 and actuation assembly 70 in the closed or locked condition. In particular, when pivot points “P1” are located radially inward of transition lines “T1 and T2”, biasing member 490 transmits forces “F1 and F2” along respective first and second links 482 and 484 which tend to force respective first and second levers 72 and 74 radially inward.

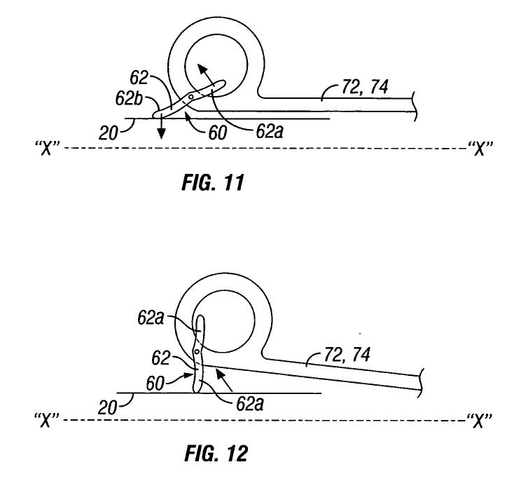

Turning now to FIGS. 11 and 12, the surgical instrument 10 may include a release trigger 60 operatively associated therewith for facilitating opening and/or unlocking of actuation assembly 70 and/or any of the latching mechanism 100 disclosed hereinabove. Release trigger 60 includes an elongated body 62 pivotally connected to at least one of first and second levers 72 and 74, preferably at or near a mid point thereof. Preferably, as seen in FIG. 11, body 62 of release trigger 60 is dimensioned such that when actuation assembly 70 or any of the latching mechanisms are in the closed condition, a first half 62a and/or a second half 62b of body 62 is contactable with a portion of surgical instrument 10 other than the lever 72 or 74 to which release trigger 60 is attached.

In use, when actuation assembly 70 or any of the latching mechanisms are in the closed condition, body 62 of release trigger 60 is pivoted about is pivot point (e.g., its mid point) such that first half 62a and/or second half 62b of body 62 contacts a portion of surgical instrument 10 other than the lever 72 or 74 to which release trigger 60 is attached. As seen in FIG. 12, as release trigger 60 is pivoted about its pivot point, first half 62a and/or second half 62b of body 62 contacts a portion of surgical instrument 10 and moves, urges or leverages first or second lever 72 or 74 away from the longitudinal “X” axis. Preferably, body 62 moves first or second lever 72 or 74 away from the longitudinal “X” axis by an amount sufficient to shift or transition the latching mechanism from the closing condition to the opening condition, as described in greater detail hereinabove.

From the foregoing and with reference to the various figure drawings, those skilled in the art will appreciate that certain modifications can also be made to the present disclosure without departing from the scope of the present disclosure. For example, it is envisioned that surgical instrument 10 may include other types of latching mechanisms which are designed to accomplish the same purpose as the latching mechanisms disclosed herein, e.g., automatic closure of actuation assembly 70 and/or end effector assembly 50.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of a preferred set of embodiments. Those skilled in the art will envision other modifications within the scope and spirit of the claims appended hereto.

What is claimed:

1. A surgical instrument comprising:
a shaft having a proximal and a distal end;
a housing connected to the proximal end of the shaft;
an actuation assembly having a first lever and a second lever, each of the first and second levers being pivotably connected to the housing;
an end effector assembly operatively connected to the distal end of the shaft, the end effector assembly having a pair of opposing jaw members;
a drive rod slidably disposed within the shaft and connected to the actuation assembly, the drive rod being operable by the actuation assembly to actuate the opposing jaw members between open and closed positions; and
a latching mechanism operatively associated with the actuation assembly and drive rod for maintaining the jaw members in the closed position.

2. The surgical instrument according to claim 1, further comprising a rotating assembly operatively associated with the drive rod for rotating the end effector assembly.

3. The surgical instrument according to claim 1, wherein the instrument may be either fully or partially disposable.

4. The surgical instrument according to claim 1, further comprising a first link and a second link, the first link having a proximal end pivotally connected to the first lever and a distal end pivotally connected to a proximal end of the drive rod, the second link having a proximal end pivotally connected to the second lever and a distal end pivotally connected to a proximal end of the drive rod.

5. The surgical instrument according to claim 4, further comprising a first stem and a second stem each connecting the proximal ends of the first and second links to proximal ends of the first and second levers respectively.

6. The surgical instrument according to claim 5, further comprising a biasing member disposed between a distal surface of a flange formed on the drive rod and a fixed proximal surface provided on one of the housing and the shaft.

7. The surgical instrument according to claim 6, wherein the biasing member is a compression spring.

8. The surgical instrument according to claim 4, further comprising a first linkage and a second linkage, the first linkage connecting the first lever with the drive rod and the second linkage connecting the second lever with the drive rod.

9. The surgical instrument according to claim 8, further comprising a release lever operatively associated with at least one of the first and second links.

10. The surgical instrument according to claim 9, wherein the release lever includes a head portion configured to be selectively engaged by a catch located adjacent to the actuation assembly when the actuation assembly is in a closed position.

11. The surgical instrument according to claim 8, wherein one of the first link and the second link has a length which is greater than the other link.

12. The surgical instrument according to claim 9, wherein the release lever includes a cantilevered arm, the arm defining a catch and configured to facilitate locking and unlocking of the apparatus.

13. The surgical instrument according to claim 5, further comprising a biasing member operatively connected between a stem extending from one of the first and second levers and a second link pivotally connected to the stem and a first link pivotally extending from the other of the first and second levers, the biasing member being configured to at least one of maintain and return the actuation assembly to the closed position.

14. The surgical instrument according to claim 13, wherein the biasing member is a tension spring.

15. A surgical instrument comprising:
a shaft having a proximal end and a distal end;
a housing connected to the proximal end of the shaft;
an actuation assembly having a first lever and a second lever, each lever being pivotably connected to the housing;
an end effector assembly operatively connected to the distal end of the shaft, the end effector assembly having a pair of opposing jaw members;
a drive rod slidably disposed within the shaft and connected to the actuation assembly, the drive rod being operable by the actuation assembly to actuate the opposing jaw members between open and closed positions;
a first link and a second link each having a distal end pivotally connected to the first and second levers respectively;
a mandrel fixedly mounted to the drive rod, proximal ends of the first and second links being pivotally attached to the mandrel; and
a biasing member configured to bias the actuation assembly to one of an open and closed condition.

16. The surgical instrument according to claim 1, further comprising a release trigger pivotally connected to at least one of the first and second levers, the release trigger configured and dimensioned to allow for the latching mechanism to transition between an open position and a closed position.

17. The surgical instrument according to claim 15, further comprising a release trigger pivotally connected to at least one of the first and second levers, the release trigger dimensioned to allow for the instrument to transition between an open position and a closed position.
